(19) United States
(12) Patent Application Publication
(10) Pub. No.: US 2004/0011357 A1
(43) Pub. Date: Jan. 22, 2004

(54) Actuator comprising moveable membrane

(76) Inventor: Philip Braithwaite, Tewkesbury (GB)

Correspondence Address: BROMBERG & SUNSTEIN LLP, 125 SUMMER STREET, BOSTON, MA, 02110-1618, US

(21) Appl. No.: 10/312,325

(22) PCT Filed: Jul. 5, 2001
(86) PCT No.: PCT/GB01/02954

(30) Foreign Application Priority Data
Jul. 5, 2000 (GB) 0016478.0

(51) Int. Cl. A61M 11/00

(52) U.S. Cl. 128/200.23; 128/203.23

(57) ABSTRACT

There is described an actuator comprising an inlet port and an out-let port and a valve member situated between the inlet and outlet ports, the valve member comprising a moveable apertured membrane such that in the open position an aperture in the membrane is coincident with the inlet and outlet ports and in the closed position the aperture is non-coincident with one or both of the inlet and outlet ports. There is also described an inhaler system using the actuator and a method of administering a medicament to a patient using such an inhaler system.

This invention relates to a novel form of valve and to devices comprising the novel valve.

More especially the invention relates to an actuator, for example a pressure activated actuator, e.g. a breath actuator, and a medicament delivery device, e.g. an inhalation device, comprising such an actuator.

It is well established that asthma and other respiratory diseases can be treated with medicaments administered by inhalation. Such medicaments may be administered in the form of a dry powder with the use of a dry powder inhaler (DPI) or in the form of a solution or suspension with the use of a pressurised metered dose inhaler (MDI). A particular problem encountered with MDI's is that considerable coordination is required for the patient to actuate the pressurised aerosol, thus dispensing the medicament, and inhaling at the correct moment. The problem is exacerbated by the fact that many patients being administered such medicaments are often children or the elderly.

Thus, there has long been a need for a simple but effective breath actuated mechanism which ensures that the patient inhales at the same time as the aerosol canister is actuated for administration of the medicament. This is often achieved by the use of a breath actuated valve situated in the inhaler or as an integral part of the inhaler. Such a valve is described, for example, in International Patent Application No WO 98/41254. The breath actuated valve described therein comprise a flexible tube which is moveable from a closed, kinked, position to an open, unkinked, position. Whilst such a valve is simple and inexpensive to manufacture, it suffers from the disadvantage that, because a solution or suspension of the medicament must pass through the valve tube, it risks becoming blocked with a build up of deposited medicament.

We have now found a novel form of actuator which is especially suitable for use as a breath activated actuator valve in an inhaler eg an MDI. However, the valve actuator does have greater utility and may be adapted for use in numerous settings eg conventionally known oil, gas or water pipes.

Thus according to the invention we provide an actuator comprising an inlet port and an outlet port and a valve member situated between the inlet and outlet ports, the valve member comprising a moveable apertured membrane such that in the open position an aperture in the membrane is coincident with the inlet and outlet ports and in the closed position the aperture is non-coincident with one or both of the inlet and outlet ports.

In some actuator mechanisms of the invention it is conceivable that the moveable apertured membrane is mechanically or electromechanically moved. However, in the most preferred embodiment the membrane is moved by the creation of a pressure differential on either side of the membrane. For example, in the case of a breath activated actuator valve, the pressure differential is created by the patient inhaling.

Thus according to a preferred embodiment we provide an actuator mechanism as hereinbefore described wherein the membrane is moveable from the closed to the open position, or vice versa, by the creation of a pressure differential across the membrane between the inlet and the outlet ports.

The actuator mechanism may comprise a membrane situated at one side of an expansion chamber, such that when the pressure differential is applied across the membrane, the membrane is moved from the closed position to the open position. The expansion chamber comprises a first wall and a second wall, the second wall being of greater dimensions eg of greater surface area, than the first wall. The dimensions of the membrane will be such as to be similar to the dimensions of the second wall ie greater than the first wall. Thus, in the closed position a portion of the membrane will lie outside the expansion chamber. In the closed position the membrane is held substantially against the first wall, such that any apertures in the membrane are held and sealed against the wall. When the membrane moves to the open position, the applications of the pressure differential urges the membrane to move from the first wall and therefore exposing the apertures and allowing flow of material through the inlet and outlet ports via the apertures. If the pressure. differential applied is sufficient, then the membrane may be urged to lie substantially against the second wall, in which the apertures will be arranged so as to be coincident with the inlet and outlet ports. Therefore, a portion of the membrane outside the expansion chamber in the closed position, will be drawn into the expansion chamber in the open position.

In the most preferred embodiment the expansion chamber comprises a hemispherical chamber wherein the second wall is hemispherical or arcuate and the first wall is plane wall. The hemispherical wall is provided with an outlet port and the plane wall is provided with an inlet port.

The membrane is anchored at one end and is provided with biasing means at the other end, keeping the membrane taught against the plane wall of the expansion chamber.

Thus the pressure differential applied across the membrane preferably provides a greater pressure on the side of the membrane adjacent the plane first wall than that experienced on the side adjacent the arcuate second wall.

The pressure differential can be applied by increasing the pressure on the plane first wall side or by decreasing the pressure on the arcuate second wall side. In the case of a breath actuated valve, the pressure differential is created by the patient inhaling, thus decreasing the pressure on the arcuate second wall side of the membrane.

As previously mentioned, the actuator mechanism of the invention has utility in a variety of areas. However, it is most suitable for use in a medicament delivery device, such as an MDI, thus creating a breath actuated MDI. Therefore according to a further feature of the invention we provide a pressure activated metered dose medicament delivery device comprising an actuator mechanism as hereinbefore described.

Such a pressure activated metered dose medicament delivery device will comprise a body adapted to retain a medicament dispenser eg in the form of an aerosol canister, and a medicament delivery orifice, the actuator mechanism of the invention will generally be situated between the medicament dispenser and the medicament delivery orifice although other sitings of the actuator mechanism are possible.

In a preferred embodiment, a pressure activated medicament delivery device of the invention comprises a body and a medicament delivery orifice and situated between the body and the orifice is an actuator mechanism as hereinbefore described, but the expansion chamber is remote from the valve mechanism for example, the expansion chamber may be attached to the side of the body.

By the term pressure activated we mean activated by the creation of a pressure differential. Thus, the pressure differential may arise from an increase or a decrease in pressure, e.g. by application of a vacuum, for example, by a patient sucking or inhaling.

According to a preferred aspect of the invention we provide a breath actuated inhaler, e.g. an MDI, comprising a valve mechanism as hereinbefore described.

The preferred MDI of the invention comprises a body and a mouthpiece; situated between the body and the mouthpiece is an actuator mechanism as hereinbefore described; an expansion chamber is provided which is remote from the actuator mechanism, for example, at the side of the body.

The actuator mechanism may comprise a membrane with a single aperture or a plurality of apertures. When a plurality of apertures is used then a corresponding number of inlet and outlet ports may be present between the medicament dispenser and the mouthpiece The inlet and outlet ports may take the form of conduits of eg 1-2 mm diameter between the medicament dispenser and the mouthpiece. When a plurality of conduits are present, there may be either side of the membrane such that the membrane is sandwiched between the sets of conduits. In such a mechanism the conduits will be aligned whilst the apertures of the membrane are non-coincident with the conduits until the actuator is activated and the membrane apertures are moved to be coincident with the conduits.

The expansion chamber may advantageously be provided with one or more air inlet orifices which aid the creation of a pressure differential at either side of the membrane. The air inlet orifices should be in the planar first wall of the expansion chamber. Any number of air inlet orifices may be included, but we have found that from 1 to 6, e.g. 4, is suitable.

Preferentially, the membrane is anchored at one end and a load attached at the other end. When used in an MDI, the membrane may be anchored adjacent the dispensing conduits and loaded at the other end. However, it is preferred that the membrane is anchored at the distal end of the strip and loaded at the end adjacent to the dispensing conduits.

The mouthpiece of the inhaler may lead directly to the expansion chamber of the actuator mechanism. However, in the preferred embodiment wherein the expansion chamber is situated on the side of the MDI body one or more conduits may lead from the mouthpiece to the expansion chamber.

Many different materials may be used as the membrane in the actuator mechanism. The material may vary depending upon the nature of the material intended to pass through the valve for example, if the actuator mechanism is intended to be used in an oil pipeline, then it must be non-perishable when in contact with oil. Most importantly it should be a flexible, non-elastic and non-porous material. More particularly when the actuator mechanism is used such it is actuated by a gas pressure differential, as in a breath actuated MDI, then the membrane material should be nongas permeable. Thus, plastics materials are well suited for use as the membrane material, poly vinyl acetate being one example of such a plastics material.

A variety of medicaments may be administered by using the inhaler of the invention. Such medicaments are generally antibiotics, bronchodilators or other anti-asthma drugs. Such medicaments include, but are not limited to β₂-agonists, e.g. fenoterol, formoterol, pirbuterol, reproterol, rimiterol, salbutamol, salmeterol and terbutaline; non-selective beta-stimulants such as isoprenaline; xanthine bronchodilators, e.g. theophylline, aminophylline and choline theophyllinate; anticholinergics, e.g. ipratropium bromide; mast cell stabilisers, e.g. sodium cromoglycate and ketotifen; bronchial anti-inflammatory agents, e.g. nedocromil sodium; and steroids, e.g. beclomethasone dipropionate, fluticasone, budesonide and flunisolide; and combinations thereof

It is within the scope of this invention for two or more medicaments to be administered.

Specific combinations of medicaments which may be mentioned include combinations of steroids, such as, beclomethasone dipropionate, fluticasone, budesonide and flunisolide; and combinations of to β₂-agonists, such as, formoterol and salmeterol. It is also within the scope of this invention to include combinations of one or more of the aforementioned steroids with one or more of the aforementioned β₂-agonists.

Further medicaments which may be mentioned include systemically active materials, such as, proteinaceous compounds and/or macromolecules, for example, hormones and mediators, such as insulin, human growth hormone, leuprolide and alpha interferon; growth factors, anticoagulants, immunomodulators, cytolcines and nucleic acids.

It is within the scope of this invention to include combinations of any of the aforementioned medicaments.

According to a further aspect of the invention we provide a method of delivering a medicament which comprises the use of a pressure activated medicament delivery device as hereinbefore described.

According to a yet further aspect of the invention we provide a method of treatment of a patient suffering from a disorder, e.g. a respiratory disorder or a systemic disorder, such as insulin dependent diabetes which comprises the administration of a therapeutically effective amount of a medicament by the use of a pressure activated medicament delivery device as hereinbefore described.

Thus we especially provide a method as hereinbefore described wherein the disorder is insulin dependant diabetes and the medicament is insulin.

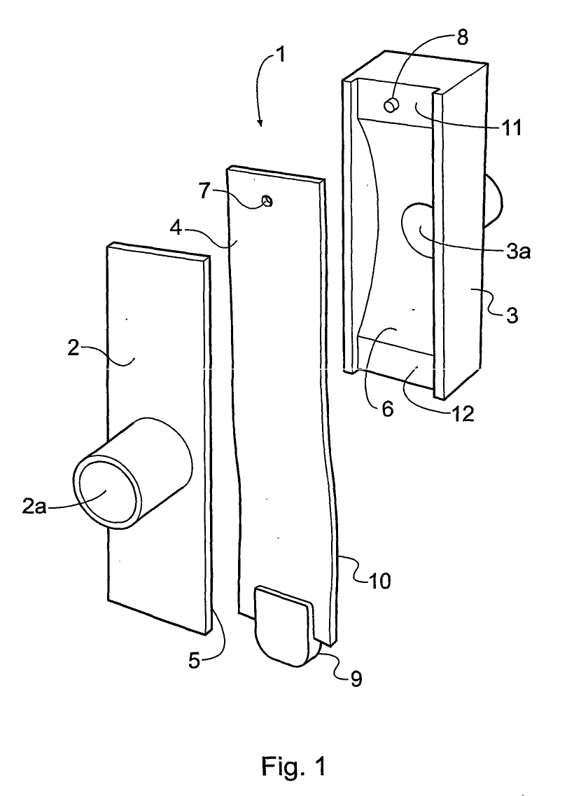

The invention will now be described by way of example only and with reference to the accompanying drawings, in which FIG. 1 is a perspective drawing of a disassembled valve of the invention;

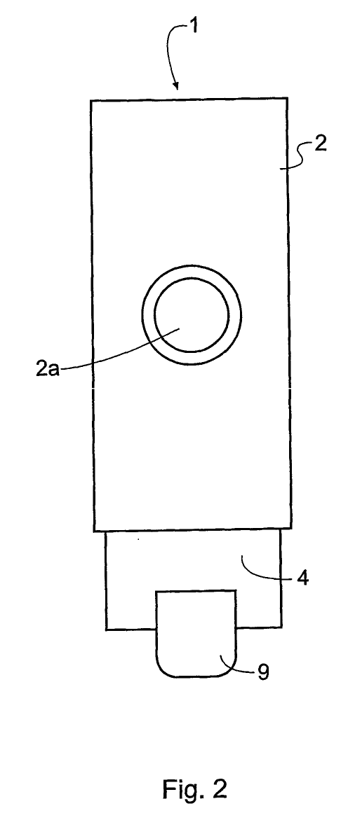

FIG. 2 is an end view of a valve of the invention;

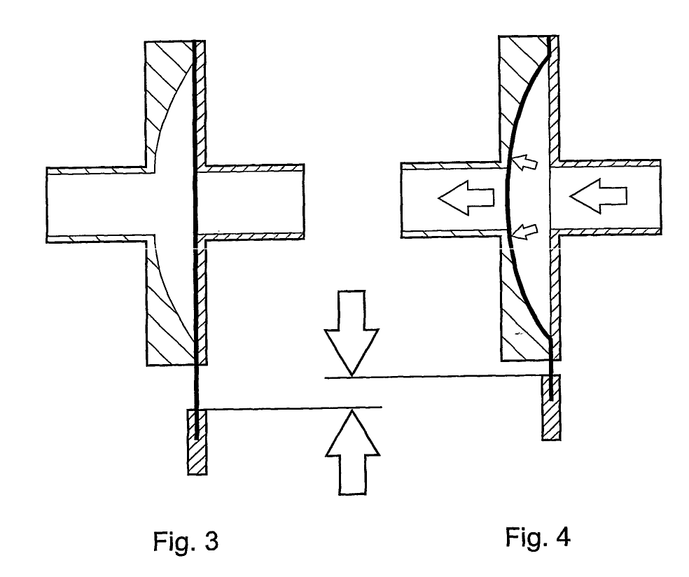

FIG. 3 is a cross-sectional side view of valve of the invention in the closed position;

FIG. 4 is a cross-sectional side view of a valve of the invention in the open position;

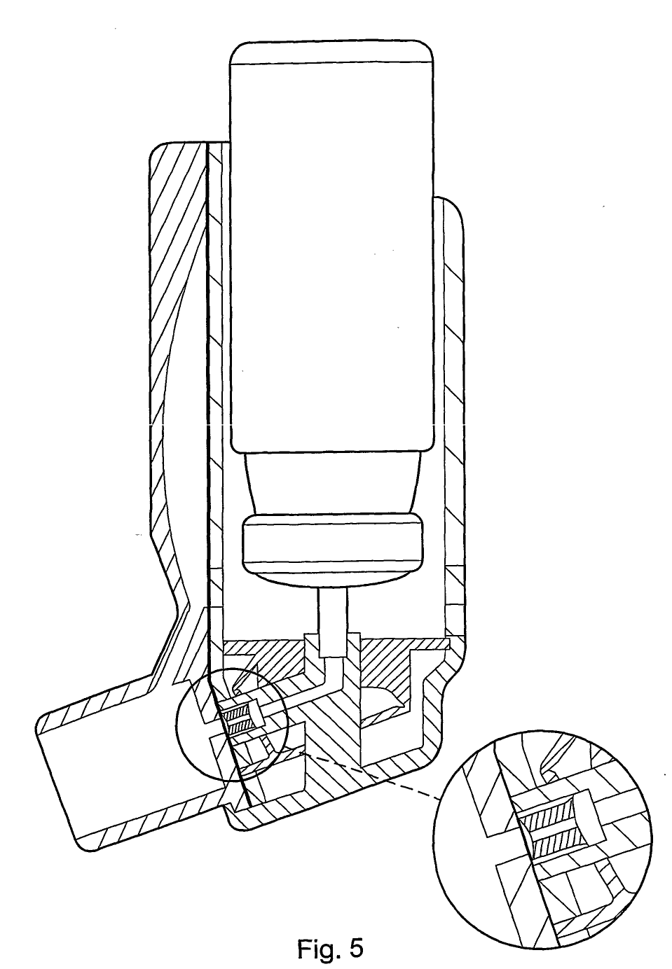

FIG. 5 is a cross-sectional side view of a metered dose inhaler comprising a valve of the invention;

FIG. 6 is a schematic partial representation of a metered dose inhaler comprising a valve of the invention in the closed position; and

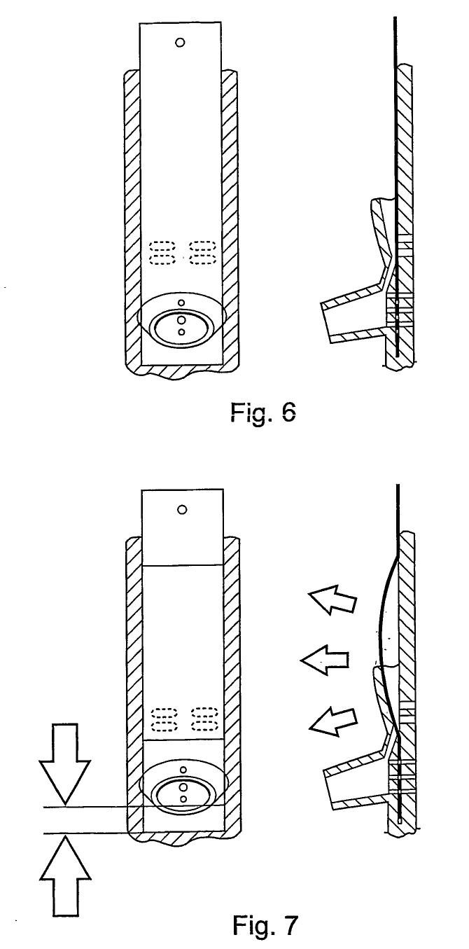

FIG. 7 is a schematic partial representation of a metered dose inhaler comprising a valve of the invention in the open position.

Referring to FIGS. 1 and 2, an actuator mechanism (1) comprises an inlet port member (2) with an inlet conduit (2a) and an outlet port member (3) with an outlet conduit (3a), and a membrane (4). The inner surface(s) of the inlet port member (2) is substantially planar and the inner surface (6) of the outlet port member (3) is arcuate. The membrane (4) is provided with an anchorage point (7) in the form of an aperture which is adapted to engage with an anchoring point (8) in the form of a protrusion on the inner surface (6) of the outlet port member (3). In order to retain the membrane (4) taut, a biasing weight (9) is attached to the end (10) of the membrane (4) distal to the anchorage point (7). Also, the inner arcuate surface (6) of the outlet port member (3) has a planar surface (11) adjacent the anchoring point (8). A planar surface (12) is also provided at the end of the arcuate surface distal to the anchoring point (8). Thus, when the inlet port member (2) and the outlet port member (3) are brought together, the membrane (4) is held against the inner planar surface (6) of the inlet port (2).

Referring to FIGS. 3 and 4, when the actuator mechanism is the closed position, the membrane (4) is held against the inner planar surface (5) of the inlet port member (2) by the planar portions (11 and 12) of the outlet port member (3).

The inlet port member (2) and the outlet port member (3) join together and the inner planar surface (5) and the inner arcuate surface (6) create an expansion chamber (13).

The biasing member (9) is attached to the end (10) of the membrane (4) and keeps. the membrane (4) taut. When a pressure differential is applied across the membrane (4), for example a lower pressure is exerted on the outlet port (3), leaving a (relatively) higher pressure on inlet port (2), then the film material is urged from the closed position, to lie against the inner arcuate surface (6) of the outlet port member (3). Since the membrane (4) is anchored around it's anchorage point (7), the distal end (10) of the membrane (4) is urged into the expansion chamber (13).

Referring to FIG. 5, a metered dose inhaler (MDI) (14) comprises a body (15), adapted to house a medicament containing aerosol (16), and a mouthpiece (17). The body (15) is provided on one side (18) with an expansion chamber (19) which is connected to the mouthpiece (17) via a conduit (20). The expansion chamber (19) comprises an inner arcuate surface (22) and on the side adjacent to the body (15) it has a planar surface (21). A membrane (23) lies adjacent the planar surface (21) and is anchored about a point (24) between a planar end surface (25) of the arcuate member (26). The membrane (23) is kept taught by squeezing between planar surfaces (27 and 28) and the inner portion of the mouthpiece (17).

Referring to FIG. 6, the inner end of the mouthpiece (17) is provided with conduits (29) connecting the mouthpiece (17) to the medicament dispensing chamber (30). A membrane (23) is anchored against the planar surface (21) of the expansion chamber (19). Apertures (33) in the membrane are misaligned with the conduits (2a). The conduits (29) are made up of two parts, a first part (31) adjacent to the mouthpiece (17) and a second part (32) adjacent to the medicament dispensing chamber (30). Apertures (33) in the membrane are misaligned with the conduits (29) and the membrane (23) is held taught by being squeezed between the first and second ports (31 and 32) of the conduit member. In this position, the valve mechanism is closed. Although not essential, the expansion chamber (19) is provided with air inlets (34) to facilitate creation of a pressure differential.

Referring to FIG. 7, when a patient depresses the medicament containing aerosol (16) (not shown) medicament is released, but with the valve in the closed position, no medicament is dispensed. When a patient inhales through the mouthpiece (17) a pressure differential is created across the membrane (23). This is facilitated by the presence of air inlets (33). The membrane (23) is urged against the arcuate surface (6) of the expansion chamber (19). This causes the distal end (10) of the membrane (23) to be pulled towards the expansion chamber (19). Therefore, the apertures (33) of the membrane (23) are brought into line with the conduits (29) and thus bringing the valve mechanism into the open position and allowing the free flow of medicament.

1. An actuator comprising an inlet port and an outlet port and a valve member situated between the inlet and outlet ports, the valve member comprising a moveable apertured membrane such that in the open position an aperture in the membrane is coincident with the inlet and outlet ports and in the closed position the aperture is non-coincident with one or both of the inlet and outlet ports.

2. An actuator according to claim 1 wherein the membrane is moved by the creation of a pressure differential on either side of the membrane.

3. An actuator according to claim 2 wherein the pressure differential is created by a patient inhaling.

4. An actuator according to claim 2 wherein the membrane is moveable from the closed to the open position, or vice versa, by the creation of a pressure differential across the membrane between the inlet and the outlet ports.

5. An actuator according to claim 1 wherein the valve mechanism comprises a membrane situated at one side of an expansion chamber, such that when the pressure differential is applied across the membrane, the membrane is moved from the closed position to the open position.

6. An actuator according to claim 5 wherein the expansion chamber comprises a first wall and a second wall, the second wall being of greater dimensions than the first wall.

7. An actuator according to claim 1 wherein, in the closed position a portion of the membrane lies outside the expansion chamber.

8. An actuator according to claim 5 wherein the expansion chamber comprises a hemispherical chamber wherein the second wall is hemispherical or arcuate and the first wall is plane wall.

9. An actuator according to claim 5 wherein the membrane is anchored at one end and is provided with biasing means at the other end, keeping the membrane taut against the plane wall of the expansion chamber.

10. An actuator according to claim 5 wherein the pressure differential applied across the membrane provides a greater pressure on the side of the membrane adjacent the plane first wall than that experienced on the side adjacent the arcuate second wall.

11. An actuator according to claim 1 wherein the pressure differential is applied by decreasing the pressure on the arcuate second wall side.

12. A pressure activated medicament delivery device comprising an actuator mechanism according to claim 1

13. A pressure activated medicament delivery device according to claim 12 comprising a body adapted to retain a medicament dispenser, a medicament delivery orifice and an actuator mechanism according to claim 1.

14. A pressure activated medicament delivery device according to claim 13 wherein the actuator mechanism is situated between the body and the medicament delivery orifice and the expansion chamber is remote from the actuator mechanism.

15. A pressure activated medicament delivery device according to claim 14 wherein the expansion chamber may be attached to the side of the body of the device.

16. A pressure activated medicament delivery device according to claim 12 characterised in that the device is an inhaler.

17. A pressure activated medicament delivery device according to claim 16 characterised in that the inhaler is a breath actuated inhaler.

18. A pressure activated medicament delivery device according to claim 16 characterised in that the inhaler is an MDI.

19. A pressure activated medicament delivery device according to claim 13 wherein the actuator mechanism comprises a membrane with aplurality of apertures.

20. A pressure activated medicament delivery device according to claim 19 wherein the number of inlet and outlet ports corresponds to the number of apertures.

21. A pressure activated medicament delivery device according to claim 13 wherein the expansion chamber is provided with one or more air inlet orifices.

22. A pressure activated medicament delivery device according to claim 13 wherein the membrane is anchored at the distal end of the strip and loaded at the end adjacent to the dispensing conduits.

23. A pressure activated medicament delivery device according to claim 15 wherein one or more conduits lead from the mouthpiece to the expansion chamber.

24. A method of delivering a medicament which comprises the use of a pressure activated medicament delivery device according to claim 12.

25. A method of treatment of a patient suffering from a disorder which comprises the administration of a therapeutically effective amount of a medicament by the use of a pressure activated medicament delivery device according to claim 12.

26. A method according to claim 25 characterised in that the disorder is insulin dependant diabetes and the medicament is insulin.

27. An actuator or a pressure activated medicament delivery device substantially as described with reference to the accompanying examples and drawings.
